SEQUENCE LISTING (6 sequences)

SEQ ID NO: 1 - DNA, 20 bp, Artificial Sequence
  PCR primer
ctgccaggcatctttctagc

SEQ ID NO: 2 - DNA, 20 bp, Artificial Sequence
  PCR primer
ttctgcaggttggaggcaac

SEQ ID NO: 3 - DNA, 20 bp, Artificial Sequence
  PCR primer
ctgctctttactgaaggctc

SEQ ID NO: 4 - DNA, 20 bp, Artificial Sequence
  PCR primer
gcctagccgagggagagccg

SEQ ID NO: 5 - DNA, 22 bp, Artificial Sequence
  PCR primer
tgtgacttgggagctctgcagc

SEQ ID NO: 6 - DNA, 18 bp, Artificial Sequence
  PCR primer
gccgccccgactgcatct

(19) United States
(12) Patent Application Publication
(10) Pub. No.: US 2008/0268478 A1
(43) Pub. Date: Oct. 30, 2008

(54) Use of Eotaxin as a Diagnostic Indicator For Atherosclerosis and Vascular Inflammation

(75) Inventors: Behrooz Sharifi, Woodland Hills, CA (US); Prediman K. Shah, Los Angeles, CA (US); Lai Wang, La Verne, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 11/817,209

(22) PCT Filed: Feb. 28, 2006
(86) PCT No.: PCT/US06/07018

§ 371 Date: Aug. 27, 2007

Related U.S. Application Data
(60) Provisional application No. 60/657,627, filed on Mar. 1, 2005.

(51) Int. Cl. G01N 33/53; G01N 33/543; C40B 30/04

(52) U.S. Cl. 435/7.92; 435/7.8; 435/7.1; 506/9

(57) ABSTRACT

The invention disclosed herein relates to the detection or diagnosis of atherosclerosis by measuring the level of the protein eotaxin in an individual's serum. The presence of eotaxin above levels specified herein is indicative that atherosclerosis may be present. Detection of elevated eotaxin levels in serum may provide a means to diagnose atherosclerosis prior to the onset of symptoms.

### FIELD OF THE INVENTION

The invention relates, in general, to the detection and monitoring of atherosclerosis and vascular inflammation.

### BACKGROUND OF THE INVENTION

Coronary heart disease is the leading cause of death in the U.S., and the leading cause of death associated with smoking. Atherosclerosis is just one of several types of arteriosclerosis, which is characterized by thickening and hardening of artery walls. More than 61 million Americans suffer from some form of cardiovascular disease, including high blood pressure, coronary heart disease, stroke, congestive heart failure, and other conditions. More than 2,600 Americans die every day because of cardiovascular diseases; about 1 death every 33 seconds.

One of the problems associated with atherosclerosis is the difficulty in early detection of the disease; indeed, atherosclerosis often shows no symptoms until flow within a blood vessel has become seriously compromised. Typical symptoms of atherosclerosis include chest pain when a coronary artery is involved, or leg pain when a leg artery is involved. However, there is no convenient blood test or similar diagnostic tool that can be used to assess the presence or pathology of this disease condition, nor is there such a means for monitoring disease progression. Current methodologies for diagnosing or monitoring atherosclerosis include identifying an abnormal difference between the blood pressure of the ankle and arm (i.e., the ankle/brachial index, or “ABI”), Doppler study of the affected area, Ultrasonic Duplex scanning, CT scan of the affected area, magnetic resonance arteriography (“MRA”), arteriography of the affected area, intravascular ultrasound (“IVUS”) of the affected vessels and cardiac stress testing. However, these techniques are typically performed only after a patient reports with symptoms. It would be beneficial for health care practitioners to have a simple, accurate means for diagnosing atherosclerosis and/or vascular inflammation, for monitoring treatment of the same, and/or for monitoring recurrence of the same.

There is a need in the art for a convenient diagnostic tool and a means for monitoring progression of disease in patients with atherosclerosis. In particular, a clinical diagnostic tool employing a serum marker—whether embodied in a kit or otherwise—would be of immense utility in the diagnosis and treatment of this disease. In patients that have a hereditary risk for coronary heart disease or for whom there are environmental factors that predispose them to the disease condition, a sensitive and specific serum test for eotaxin might obviate the need for treatment.

Additionally, when atherosclerosis is diagnosed, there is no means by which to assess the impact (positive or negative) of therapeutic intervention, or to monitor recurrence, other than with intrusive tests. The ability to derive this information by studying a serum marker would establish a means of detecting recurrence earlier in disease progression than that which is possible by use of conventional methods of diagnosis.

### SUMMARY OF THE INVENTION

The invention disclosed herein provides compositions and methods useful for detecting the presence of atherosclerosis in a mammalian subject. In accordance with certain embodiments of the invention, methods comprise obtaining a sample of serum from a mammalian subject, contacting the serum with an eotaxin binding partner, whereby an eotaxin/eotaxin binding partner complex is formed, detecting the eotaxin/eotaxin binding partner complex, wherein the level of eotaxin/binding partner complex correlates with the eotaxin serum level of the subject, and correlating the serum level of eotaxin with the presence of atherosclerosis.

Further embodiments include methods wherein the binding partner is selected from the group consisting of peptides, antibodies, small molecules, and combinations thereof, as well as embodiments wherein the binding partner is detectably labeled.

Still further embodiments provide methods wherein the detecting of the eotaxin/eotaxin binding partner complex is accomplished by enzyme-linked immunosorbent assay (ELISA) or by antibody array.

Other embodiments provide methods wherein the eotaxin/eotaxin binding partner complex is detected by a second binding partner.

Additional embodiments of the invention provide methods useful for diagnosing atherosclerosis in a mammal, comprising obtaining a sample of serum from a mammalian subject, contacting the serum sample with an eotaxin binding partner capable of forming a complex with eotaxin, and detecting the presence of the eotaxin/eotaxin binding partner complex, wherein the concentration of the eotaxin/eotaxin binding partner complex correlates with the concentration of eotaxin in the serum of the mammalian subject, and wherein a concentration of eotaxin greater than about 160 pg/mL indicates that the mammal has atherosclerosis.

Other embodiments of the invention provide kits for the diagnosis of atherosclerosis in a mammalian subject, comprising a solid substrate having immobilized thereon a first anti-eotaxin antibody, second anti-eotaxin antibody reactive with the first anti-eotaxin antibody, wherein the second anti-eotaxin antibody is detectably labeled, instructions for performing an enzyme-linked immunosorbent assay (ELISA) for the presence of eotaxin in a sample of serum obtained from the mammalian subject; and one or more reagents for performing the ELISA.

Further embodiments comprise kits comprising a multi-well microtiter plate, and wherein the first antibody is immobilized in the wells of the multi-well microtiter plate as well as kits wherein the second antibody is fluorescently-labeled.

### BRIEF DESCRIPTION OF THE DRAWINGS

FIG. 1 shows the plasma levels of eotaxin in ApoE and ApoE/TN mice fed on a high-fat diet at 1 and 3 weeks using an ELISA procedure in accordance with an embodiment of the present invention.

FIG. 2 shows the plasma levels of eotaxin in ApoE and TN/E mice fed on a normal diet at 6, 8, 9 and 10 weeks using an ELISA procedure in accordance with an embodiment of the present invention.

FIG. 3 shows the template of 62 mouse proinflammatory molecules including positive controls on the antibody array in accordance with an embodiment of the present invention.

FIG. 4 shows a representative membrane image from the ApoE group and ApoE/TN groups on a high fat diet for 24 weeks in accordance with an embodiment of the present invention.

FIG. 5 shows plasma eotaxin levels in ApoE/TN and ApoE mice measured by ELISA in accordance with an embodiment of the present invention.

FIG. 6A shows the expression of TN in a lesion of an ApoE null mouse that were placed on high-fat diet (10 mice/group) for 4 weeks in accordance with an embodiment of the present invention.

FIG. 6B shows the expression of TN in a lesion of an ApoE null mouse that were placed on high-fat diet (10 mice/group) for 8 weeks in accordance with an embodiment of the present invention.

FIG. 6C shows the expression of TN in a lesion of an ApoE null mouse that were placed on high-fat diet (10 mice/group) for 20 weeks in accordance with an embodiment of the present invention.

FIG. 7A shows a representative photograph of the gross appearance of the aortic arch from an ApoE null mouse in accordance with an embodiment of the present invention.

FIG. 7B shows a representative photograph of the gross appearance of the aortic arch from an ApoE/TN null mouse in accordance with an embodiment of the present invention.

FIG. 8A shows the aortic arch that was exposed and photographed under a dissecting microscope with appropriate back lighting in accordance with an embodiment of the present invention. Panel A shows the distribution of aortic lesions from the ApoE mouse group on a high-fat diet for 18 weeks.

FIG. 8B shows the aortic arch that was exposed and photographed under a dissecting microscope with appropriate back lighting in accordance with an embodiment of the present invention. Panel B shows the distribution of aortic lesions from the ApoE/TN mouse group on a high-fat diet for 18 weeks.

FIG. 8C shows an aorta that was exposed and photographed under a dissecting microscope with appropriate back lighting in accordance with an embodiment of the present invention. Panel C shows an Oil red 0 staining of an aorta from the ApoE mouse group.

FIG. 8D shows an aorta that was exposed and photographed under a dissecting microscope with appropriate back lighting in accordance with an embodiment of the present invention. Panel C shows an Oil red 0 staining of an aorta from the ApoE/TN mouse group.

FIG. 9 shows the relative lesion area in ApoE/TN and ApoE mice on high-fat diet for 4-20 weeks in accordance with an embodiment of the present invention.

FIG. 10 shows the aortic arch from ApoE/TN null mice placed on high-fat diet for 30 weeks and then sacrificed in accordance with an embodiment of the present invention.

FIG. 11 shows the H&E staining of aortic lesion of TN/E mice fed a high-fat diet for 30 weeks at 20× magnification in accordance with an embodiment of the present invention.

FIG. 12 shows the toluidine blue staining of aortic lesion of TN/E mice fed a high-fat diet for 30 weeks at 10× magnification in accordance with an embodiment of the present invention.

FIG. 13A shows mast cell accumulation in the aorta of TN/E mice fed a high-fat diet for 30 weeks at 100× magnification in accordance with an embodiment of the present invention.

FIG. 13B shows mast cell accumulation in the aorta of TN/E mice fed a high-fat diet for 30 weeks at 10× magnification in accordance with an embodiment of the present invention.

FIG. 14A shows lesion development in the aortic sinuses of an ApoE null mouse on a high-fat diet for 4 weeks in accordance with an embodiment of the present invention.

FIG. 14B shows lesion development in the aortic sinuses of an ApoE/TN null mouse on a high-fat diet for 4 weeks in accordance with an embodiment of the present invention.

FIG. 14C shows macrophage distribution in an ApoE/TN null mouse on high-fat diet for 4 weeks in accordance with an embodiment of the present invention.

FIG. 15A shows a representative Oil red 0 staining of the frozen sections from innominate artery of an ApoE null mouse on high-fat diet for 4 weeks.

FIG. 15B shows a representative Oil red 0 staining of the frozen sections from innominate artery of an ApoE/TN null mouse on high-fat diet for 4 weeks in accordance with an embodiment of the present invention.

FIG. 15C shows Oil red O staining of frozen section from artery of ApoE null mice on high-fat diet for 18 weeks in accordance with an embodiment of the present invention.

FIG. 15D shows a representative Oil red O staining of left subclavian artery from ApoE/TN null mice on high-fat diet for 4 weeks in accordance with an embodiment of the present invention.

FIG. 16A shows the distribution of SMCs in the innominate artery lesions of a ApoE null mouse in accordance with an embodiment of the present invention.

FIG. 16B shows the distribution of SMCs in the innominate artery lesions of a ApoE/TN null mouse in accordance with an embodiment of the present invention.

FIG. 17A shows total serum cholesterol measured in serum from 5 individual ApoE and ApoE/TN null mice on high-fat diet for 4-20 weeks in accordance with an embodiment of the present invention.

FIG. 17B shows lipoprotein-cholesterol distribution measured in serum from 5 individual ApoE and ApoE/TN null mice using superose-6 chromatography in accordance with an embodiment of the present invention.

FIG. 18A shows the culture of aortic SMCs from ApoE null mice in accordance with an embodiment of the present invention.

FIG. 18B shows the culture of aortic SMCs from ApoE/TN null mice in accordance with an embodiment of the present invention.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994); March, Advanced Organic Chemistry Reactions, Mechanisms and Structure 4th ed., J. Wiley & Sons (New York, N.Y. 1992); and Sambrook and Russell, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Conditions” and “disease conditions,” as used herein may include, but are in no way limited to any form of coronary heart disease and/or its pathology; in particular, atherosclerosis and vascular inflammation.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Pathology” of coronary heart disease includes all phenomena that compromise the well-being of the patient. This includes, without limitation, all forms of arteriosclerosis, including atherosclerosis, vascular inflammation, release of cytokines or other secretory products at abnormal levels, suppression or aggravation of inflammatory or immunological response, etc.

The term “atherosclerosis” refers to a condition that results from the gradual build-up of fatty substances, including cholesterol, on the walls of the arteries. This build-up, called plaque, reduces the blood flow to the heart, brain and other tissues and can progress to cause a heart attack or stroke. Atherosclerosis is the deposition of a fatty material, called plaque, on blood vessel walls leading to narrowing of blood vessels and obstruction of blood flow to the heart, brain, and limbs. Plaque is a combination of cholesterol, fatty acids, calcium, scar tissue, and blood components that stick to the inside of the arterial wall. Some plaques are unstable and can rupture or burst, leading to blood clotting inside the artery. If a blood clot blocks an artery completely, blood flow may be stopped, which results in heart attack and/or stroke.

The invention disclosed herein is based on the surprising discovery that elevated circulating levels of the chemokine eotaxin are present in the serum of individuals with atherosclerosis. Thus, in various embodiments, the invention relates to diagnostic methods, tools, kits and other mechanisms that make use of eotaxin as a serum marker for atherosclerosis and vascular inflammation.

The observation that eotaxin levels are elevated in individuals with atherosclerosis arose from research using mice lacking the genes encoding ApoE and tenascin-C (TN). For the purposes of this application, a mouse that has been genetically modified to lack the gene encoding apolipoprotein E (ApoE) may be referred to as an “ApoE null mouse”, an “ApoE knockout” mouse, or an “ApoE mouse”. Similarly a mouse that has been genetically modified to lack the gene encoding tenascin (TN) may be referred to as a “TN null mouse”, a “TN knockout” mouse, or a “TN mouse”. Also, mice lacking both the ApoE gene and the TN gene may be referred to as “double knockout mice”, ApoE/TN null mice, ApoE/TN mice, or TN/E mice.

TN exhibits in vitro activities that are important in the pathogenesis of atherosclerotic vascular disease, and ApoE null mice lacking the TN gene were generated in order to test the hypothesis that the genetic deletion of TN in the ApoE null background modifies the development of atherosclerosis. The data show that in ApoE/TN null mice, atherosclerotic lesions developed earlier and with an increased number of inflammatory cell components, when compared to the lesions in ApoE mice. These events occurred without any significant difference in the lipoprotein profile between the two mouse genotypes. In these studies, it was found that ApoE/TN null mice were associated with markedly increased circulating levels of the chemokine eotaxin.

TN is a large modular extracellular matrix protein with complex interactions with cells (for general discussion, see Jones, F. S. and Jones, P. L., Dev Dyn 218:235-25, 2000.). Structurally, the TN polypeptide is divided into four regions, each of which appears to have a distinct biological function. Multiple functions have been attributed to TN, based upon its effects observed in cell culture and its distribution in tissues undergoing active restructuring. In different experimental models and tissues, TN has been reported to mediate cell adhesion (Grumet, M. et al., J Biol Chem 269:12142-12146, 1994., LaFleur, D. W. et al., J Biol Chem 272:32798-32803, 1997.) or to inhibit it (Chiquet-Ehrismann, R. et al., Cell 53:383-390, 1988., LaFleur, D. W. et al., J Biol Chem 269:20757-20763, 1994.), to induce differentiation (Mackie, E. J. et al., Differentiation 37:104-114, 1988.) or to inhibit it (Crossin, K. L. and Hoffman, S., Dev Biol 145:277-286, 1991.), and to stimulate cell growth (Jones, P. L. et al., Am J Pathol 150:1349-1360, 1997.) or to promote apoptosis (Boudreau, N. et al., Science 267:891-893, 1995., Wallner, K. et al., Arterioscler Thromb Vasc Biol 24:1416-1421, 2004.).

Using cultured rat and human smooth muscle cells (SMCs) as a target cells, it has been reported that TN promotes cell migration and viability. TN expression is regulated by angiotensin II and platelet-derived growth factor (LaFleur, D. W. et al., J Biol Chem 269:20757-20763, 1994., Sharifi, B. G. et al., J Biol Chem 267:23910-23915, 1992.), the alternatively spliced variant that is expressed by cultured rat and human smooth muscle cells has been identified and cloned (LaFleur, D. W. et al., J Biol Chem 269:20757-20763, 1994.). Also, the principal domain of TN that interacts with cultured SMCs (LaFleur, D. W. et al., J Biol Chem 272:32798-32803, 1997.) has been identified. With respect to the expression of TN in vivo, it was shown that arterialization of human vein grafts is associated with TN expression (Wallner, K. et al., J Am Coll Cardiol 34:871-875, 1999.), and that TN is expressed in the macrophage-rich region of human coronary atherosclerotic plaques (Wallner, K. et al., Circulation 99:1284-1289, 1999.). Further, it was found that adventitial remodeling after angioplasty is associated with the expression of tenascin mRNA by adventitial myofibroblasts, (Wallner, K. et al., J Am Coil Cardiol 37:655-661, 2001.), and that balloon catheterization induces arterial expression of a new alternatively spliced TN isoform (Wallner, K. et al., Atherosclerosis 161:75-83, 2002.). Jones et al reported that TN promotes survival of cultured SMCs (Jones, P. L. et al., J Cell Biol 139:279-293, 1997.). It was recently found that TN undergoes degradation in vivo and in vitro and that the EGF-like domain of TN has proapoptotic activity for cultured SMCs (Wallner, K. et al., Arterioscler Thromb Vasc Biol 24:1416-1421, 2004.). As already described, TN is involved in multiple important biological activities that are relevant to cardiovascular diseases. The availability of TN null mice and ApoE/TN double knockout mice have allowed the biology of TN in vascular tissue to be examined in new ways.

TN knockout mice have been developed previously (Saga, Y. et al., Genes Dev 6:1821-1831, 1992., Forsberg, E. et al., Proc Natl Acad Sci U S A 93:6594-6599, 1996.), and are reported to be phenotypically normal, fertile, and have a normal life span. Histological examinations showed no gross deficits in neuroarchitecture or principal organ systems. Hematologic parameters (hematocrit, white blood cell count, bone marrow mononuclear cell count, spleen weight, and liver weight) of TN null mice are similar to age-matched wild type C57BL/6 mice (Ohta, M. et al., Blood 91:4074-4083, 1998.). The number and size of the blood vessels of TN null mice is similar to those of wild type C57BL/6 mice (Talis 1999). Since TN belongs to family of proteins, it was thought that the absence of TN may be compensated for by up-regulation of other members of the tenascin family, such as tenascin-X or tenascin-R. However, the levels of expression of tenascin-R and tenascin-X in TN null mice is similar to wild type mice (Saga, Y. et al., Genes Dev 6:1821-1831, 1992., Steindler, D. A. et al., J Neurosci 15:1971-1983, 1995.). In addition, the expression levels of other matrix proteins such as fibronectin and laminin, and proteoglycans are not affected by TN deficiency (Talts, J. F. et al., J Cell Sci 112 (Pt 12):1855-1864, 1999.). Therefore, these mice appear to be “normal” and can be used to examine the role of TN in cardiovascular tissue.

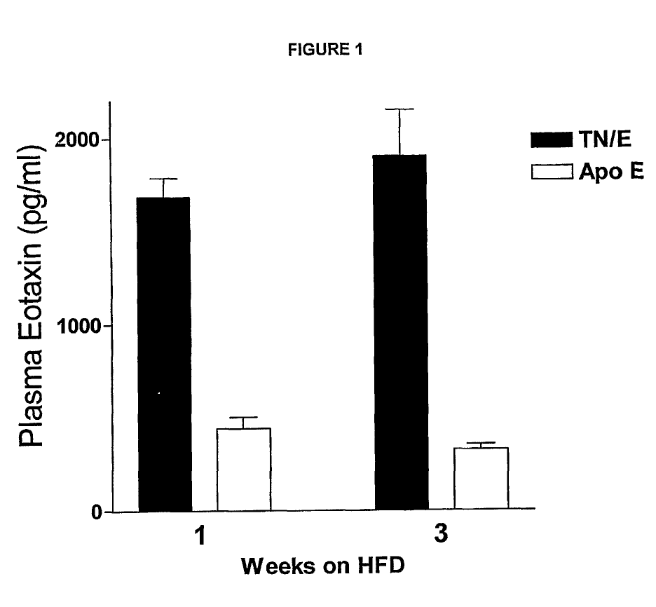

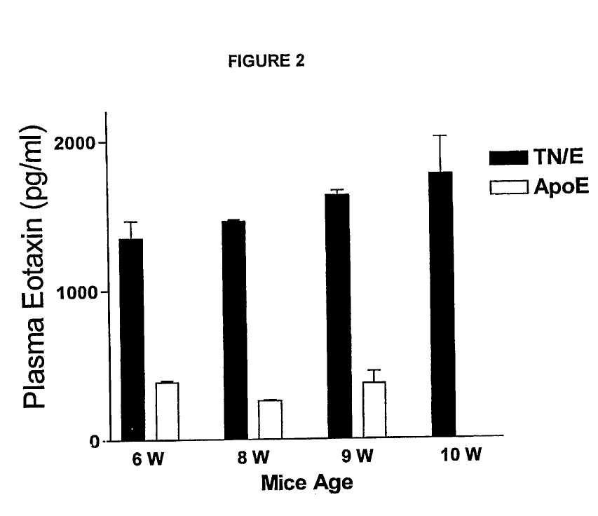

The ApoE/TN knockout mice developed by the applicants exhibit dramatic changes in the development of stenosis as well as the types of atherosclerotic lesions. Many of the details of the experiments with ApoE/TN knockout mice are outlined in Example 2. Deletion of the TN gene affects development of atherosclerosis n ApoE null mice by accelerating atherosclerotic lesion formation in ApoE/TN null mice on a high-fat diet for 4 weeks in the aortic root, the lesser curvature of the aortic arch, the principal branches of aorta, and innominate artery and increasing levels of inflammatory cells in the lesion while having no effect on hypercholesterolemia or the migration, viability, or proliferation of cultured SMCs. Ancillary to that conclusion, elevated circulating levels of the chemokine eotaxin were found in both ApoE and ApoE/TN null mice. As shown in FIGS. 1 and 2, ApoE/TN knockout mice exhibit elevated serum levels of eotaxin. Based on these findings, it is likely that ApoE/TN null mice provide a model for studying atherosclerosis in mammals. As such, the use of eotaxin as a marker for atherosclerosis and vascular inflammation is a particularly appropriate embodiment of the invention.

Eotaxin is a new member of CC family of chemokines (see Van Coillie, E. et al., Cytokine Growth Factor Rev 10:61-86, 1999. for general discussion). To date, human members of this group include MCP-1, MCP-2, MCP-3, MIP-1a, MIP-1b, RANTES, and 1-309. The eotaxin gene is well conserved in several species including human, mouse, and guinea pig. The major receptor that mediates eotaxin's biological effects is CCR3, a seven-transmembrane receptor coupled to heterotrimeric G proteins. Cell types known to produce eotaxin include endothelium (Rothenberg, M. E. et al., Natl Acad Sci U92:8960-8964, 1995.) lymphocytes, macrophages and eosinophils (Ponath, P. D. et al., J Exp Med 183:2437-2448, 1996., Ganzalo, J. A. et al., Immunity 4:1-14, 1996.).

Eotaxin plays a critical role in allergic and nonallergic inflammatory reactions, and it has been found to be overexpressed in chronic inflammatory diseases such as ulcerative colitis and Crohn's disease. In addition, eotaxin up-regulates CD11b in macrophages (Tenscher, K. et al., Blood 88:3195-3199, 1996.), modulates myelopoiesis and mast cell development from embryonic hematopoietic progenitors (Quackenbush, E. J. et al., Blood 92:1887-1897, 1998., Kempuraj, D. et al., Blood 93:3338-3346, 1999.), and promotes angiogenesis (Salcedo, R. et al., J Immunol 166:7571-7578, 2001.).

The function of eotaxin in cardiovascular disease is unclear. Gene expression profiling of an ischemic rat aortic transplant showed that eotaxin mRNA is differentially upregulated (Chen, J. et al., Am J Pathol 153:81-90, 1998.). Gene expression profiling of unstable human plaques revealed that eotaxin mRNA and protein is expressed by a small number of smooth muscle cells located around the lipid core (Haley, K. J. et al., Circulation 102:2185-2189, 2000.). Eotaxin was found to promote migration of cultured VSMC (Kodali, R. B. et al., Arterioscler Thromb Vasc Biol 24:1211-1216, 2004.). These data underscore the importance of eotaxin in vascular pathologies.

The reason for the selective over-expression of eotaxin in ApoE/TN mice is not immediately apparent. In vivo, the serum level of eotaxin is low (83+10 pg/ml) in healthy humans (Economou, E. et al., Int J Cardiol 80:55-60, 2001.). However, the level is increased in response to inflammation and infection. In addition, it is reported that eotaxin is increased in human in response to balloon angioplasty (Economou, E. et al., Int J Cardiol 80:55-60, 2001.). In ApoE and C57 mice, the level of eotaxin is low and hyperlipidemia does not significantly affect eotaxin level. In cultured cells, eotaxin is not expressed under basal, unstimulated conditions. Eotaxin mRNA and protein levels are markedly up-regulated in response to TNF-□, IL-1, IL-4, and IL-13 treatment (Chung, K. F. et al., Br J Pharmacol 127:1145-1150, 1999., Moore, P. E. et al., Am J Physiol Lung Cell Mol Physiol 282:L847-853, 2002., Matsukura, S. et al., J Immunol 163:6876-6883, 1999.). However, no difference in the plasma levels of these inflammatory factors was found between the ApoE and ApoE/TN groups (compare F9F10, J3J4, A5A6, H5H6 in FIG. 19). While the mechanism by which eotaxin is selectively upregulated in ApoE/TN mice remains to be determined, TN deficiency and chronic hyperlipidemia were found to markedly promote up-regulation of this pro-inflammatory molecule. It is possible that chronic hyperlipidemia promotes the over-expression of eotaxin in ApoE/TN mice thus destabilizing atherosclerotic plaques in these animals. Thus, research suggests that eotaxin has the ability and plays a role in selectively priming various cell types for chemotaxis, directing their migration/chemotaxis and activating inflammatory activity in the cells attracted.

However, the diagnostic potential of soluble eotaxin has heretofore remained entirely untapped. (Haley, K. J. et al., Circulation 102:2185-2189, 2000.); U.S. Pat. No. 6,548,245; U.S. patent application publication No.: 2003/0165980). As such, the use of eotaxin as a marker for atherosclerosis and vascular inflammation is a particularly appropriate embodiment of the invention.

As stated earlier, the concentration of eotaxin in the serum of a “healthy” individual (i.e., an individual without atherosclerosis) is around 80 pg/mL. The serum level of eotaxin may be considered to be elevated at levels above around 160 pg/mL. Elevated serum levels of eotaxin are an suggest that atherosclerosis may be present, and indicate that further tests for atherosclerosis may be warranted. For example, if an elevated level of eotaxin is found in a patient, follow up analysis may include identifying an abnormal difference between the blood pressure of the ankle and arm (i.e., the ankle/brachial index, or “ABI”), Doppler studies, Ultrasonic Duplex scanning, CT scans, magnetic resonance arteriography (“MRA”), arteriography, intravascular ultrasound (“IVUS”) or cardiac stress testing. It may then be possible to detect and treat the disease before any blood vessels become seriously compromised.

Various body fluids may be extracted from a subject and examined in connection with embodiments of the present invention. Such body fluids may include, but are in no way limited to, blood (including whole blood as well as its plasma and serum), urine, sweat, pulmonary secretions, tears, and a protein sample from a tumor (obtained from fresh, frozen, or paraffin embedded tumor materials); each of which is hereinafter included in the term “serum.” In a preferred embodiment, one extracts and examines a sample of blood serum from a mammalian subject.

Once a serum sample has been obtained from a mammal, it may be analyzed for the presence and concentration of eotaxin. Any conventional method may be used to screen for the presence of this chemokine, and/or to assess its concentration in the sample. In one embodiment of the invention, as described in greater detail in the ensuing Examples, an enzyme-linked immunosorbent assay (“ELISA”) procedure is utilized. In another embodiment of the invention, as described in greater detail in the ensuing Examples, a cytokine/chemokine antibody array is utilized. The ELISA procedure, the antibody array and many other conventional methodologies well known to those of skill in the art may be readily packaged or otherwise clinically or commercially assembled for use. Other methods for detection or measurement of eotaxin levels in a sample include the use fluorescently labeled antibodies that are employed in certain embodiments of the invention. Many other means of detecting eotaxin directly or detecting a complex of eotaxin with another moiety are known, including gas chromatography, mass spectroscopy, thin layer chromatography, hydroxyl apatite chromatography, high pressure liquid chromatography, colloidal gold immunolabeling read by electron microscopy, radioactively labeled tags or antibodies specific for eotaxin read using a scintillation counter, bioluminescently labeled antibodies read on a calorimeter, etc.

In certain embodiments, eotaxin is detected in a sample of blood from a mammalian subject by contacting the samples with a binding partner for eotaxin, that is, a peptide, immunoglobulin, small molecule or other moiety capable of forming an association complex with eotaxin. Upon contacting eotaxin with a binding partner, an eotaxin/eotaxin binding partner complex may be formed. Additionally, the eotaxin/eotaxin binding partner complex may be contacted with additional binding partners that recognize and/or bind to eotaxin, one of the binding partners, or the eotaxin/eotaxin partner complex. Any of the binding partners may be detectably labeled, for example, with a fluorescent tag, radioactive tag, or affinity tag. It is also considered within the scope of the invention for the binding partner to be immobilized on a solid surface, such as in a microtiter plate, or on beads. In certain embodiments of the invention, the eotaxin present in a sample may be detected using antibodies specific for eotaxin. Several such antibodies, such as those disclosed in U.S. Pat. No. 6,946,546, are known in the art. Additionally, R&D Systems supplies a number of antibodies and protocols for detecting eotaxin in mammalian serum.

The present invention is thus also directed to a kit for the diagnosis or monitoring of atherosclerosis and vascular inflammation in a subject. The kit is useful for practicing the inventive method of diagnosing atherosclerosis and vascular inflammation and/or monitoring its progression. The kit is an assemblage of materials or components, including at least one means of assessing the presence and/or level of eotaxin in the serum obtained from a subject in accordance with various embodiments of the present invention. The exact nature of the components configured in the inventive kit depends on its intended purpose and on the particular methodology that is employed. For example, some embodiments of the kit are configured for the purpose of diagnosing atherosclerosis and/or vascular inflammation in a mammalian subject. Other embodiments are configured for the purpose of determining the level or concentration of eotaxin in the serum to assess, e.g., success of a therapeutic intervention or progression of disease. In a most preferred embodiment, the kit is configured particularly for the purpose of diagnosing or monitoring human subjects.

Instructions for use may be included with the kit. “Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like, typically for an intended purpose. Optionally, the kit also contains other useful components, such as, diluents, buffers, pharmaceutically acceptable carriers, specimen containers, syringes, stents, catheters, pipetting or measuring tools, paraphernalia for concentrating, sedimenting, or fractionating samples, or antibodies and/or primers and/or probes for controls.

The materials or components assembled in the kit can be provided to the practitioner stored in any convenient and suitable ways that preserve their operability and utility. For example the components can be in dissolved, dehydrated, or lyophilized form; they can be provided at room, refrigerated, or frozen temperatures. The components are typically contained in suitable packaging material(s). As employed herein, the phrase “packaging material” refers to one or more physical structures used to house the contents of the kit. The packaging material is constructed by well known methods, preferably to provide a sterile, contaminant-free environment. The packaging materials employed in the kit are those customarily utilized in the field. As used herein, the term “package” refers to a suitable solid matrix or material such as glass, plastic, paper, foil, and the like, capable of holding the individual kit components. Thus, for example, a package can be a glass vial used to contain suitable quantities of a composition containing an antibody. The packaging material generally has an external label which indicates the contents and/or purpose of the kit and/or its components.

The tools, kits, and methods of the present invention may be implemented in connection with a diagnostic screening methodology for atherosclerosis and vascular inflammation. The various embodiments of the present invention may therefore provide a means for early detection of the aforementioned disease conditions. The embodiments of the invention are also suitable for use in connection with monitoring the success of ongoing or completed therapeutic intervention. For instance, a subject's serum may be tested prior to clinical diagnosis to screen for atherosclerosis and/or vascular inflammation; during the course of treatment (e.g., to enhance a physician's ability to implement an effective treatment regimen); and/or following the completion of an intervention to determine a level of success (e.g., lifestyle changes, angioplasty and bypass surgery).

### EXAMPLES

The following Examples illustrate a method of creating ApoE/TN null mice and performing various assays for the study of atherosclerosis; for example, an assay method for screening a serum sample for the presence and concentration of soluble eotaxin. The Examples further demonstrate that the presence of soluble eotaxin in a subject's serum may be indicative of atherosclerosis and/or vascular inflammation, and that the concentration of soluble eotaxin may correlate with disease progression. These Examples are included merely for purposes of illustration.

### Example 1

### Generation of Double Knockout Mice

TN null mice generated used in this study were healthy and pathogen-free. The TN null mouse colony is housed at the Department of Comparative Medicine and the experiments were approved by the Institutional Animal Care and Use Committee, at Cedars-Sinai Medical Center.

Male ApoE mice (C57BL/6 background) were purchased from Jackson Labs and crossed with female TN null mice (C57BL/6 background). The ApoE and TN expression were determined by polymerase chain reaction (PCR) using genomic DNA isolated from tail biopsies. The normal and mutant TN alleles were identified by PCR using three primers in a single PCR reaction. The TN upstream primer (TNUP; 5′-CTGCCAGGCATCTTTCTAGC-3′; SEQ ID NO:1) and downstream primer (TNDN; 5′-TTCTGCAGGTTGGAGGCMC-3′; SEQ ID NO:2) bind to sequences in exon 2 of the mouse TN gene and amplify a 420 bp long DNA fragment that is specific for the TN wild type allele. A third primer binds to sequences in the neo gene (NEOPA; 5′-CTGCTCTTTACTGMGGCTC-3′; SEQ ID NO:3) and together with the (downstream) TNDN primer amplifies a 340 bp fragment, which is specific for the mutant TN allele. To determine the presence or absence of the ApoE gene, 3 primers were used: MR0180 (5′-GCC TAG CCG AGG GAG AGC CG-3′; SEQ ID NO:4), MR0181 (5′-TGT GAC TTG GGA GCT CTG CAG C-3′; SEQ ID NO:5), and MR0182 (5′-GCC GCC CCG ACT GCA TCT-3′; SEQ ID NO:6). The MR0180 and MR0181 primers amplify a 155 bp wild type band, and MR0180 and MR0182 primers amplify a 245 bp band from the targeted allele. PCR reactions (25 ul) were performed for 30 cycles with 50 ng DNA, 0.2 mmol/L dNTPs, 200 pmol primers and BD Advantage 2 polymerase Mix (SD Bioscience Clontech). The TN PCR amplification profile was as follows: denaturing at 94 for 3 minutes, followed by 30 cycles at 940 C (40 seconds), 650 C (45 seconds), and 680 C (3 minutes). The offspring were divided into subgroups by their sex (male/female) and their ApoE and TN genotypes. This initial genotyping was confirmed with Southern blotting. F1 mice were inter-bred and genotyped in order to obtain homozygous ApoE/TN double knockout mice.

### Example 2

### Genetic Ablation of TN Promotes Initiation of Plaque Development in ApoE Mice

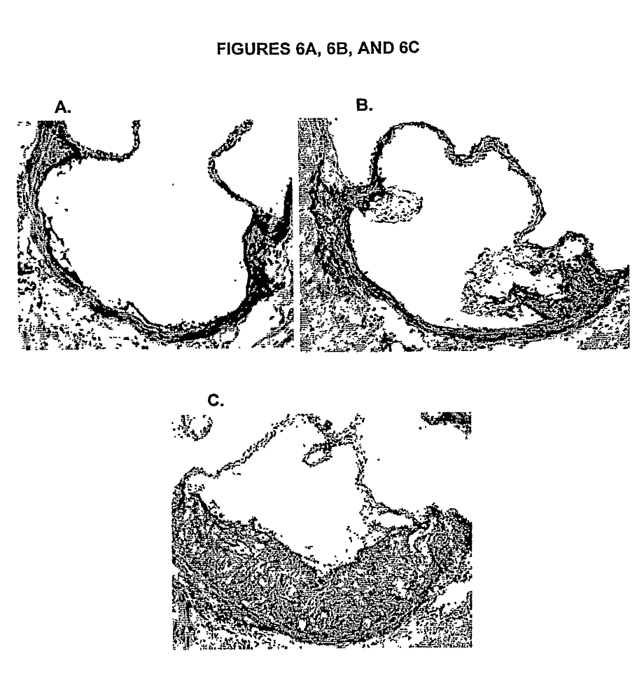

It was previously reported that TN is expressed in human atherosclerotic plaques (Wallner, K. et al. (1999)(2)). The timing of TN expression was studied in ApoE null mice on high-fat diet for 4 and 18 weeks. Consistent with previous data on the expression of TN in normal human and rat arteries (Wallner, K. et al. (2001); Wallner, K. et al. (2002); Wallner, K. et al. (1999)(1); Wallner, K. et al. (1999)(2)), TN immunoreactivity was not found in the aortic sinuses of ApoE mice on a normal diet (not shown). However, TN expression was detected in ApoE null mice on high-fat diet for 4 weeks (FIG. 6A) and the level of expression increased as lesion grew (FIG. 6C).

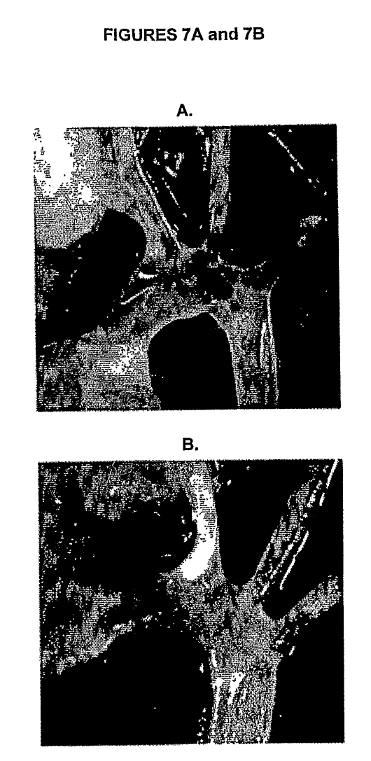

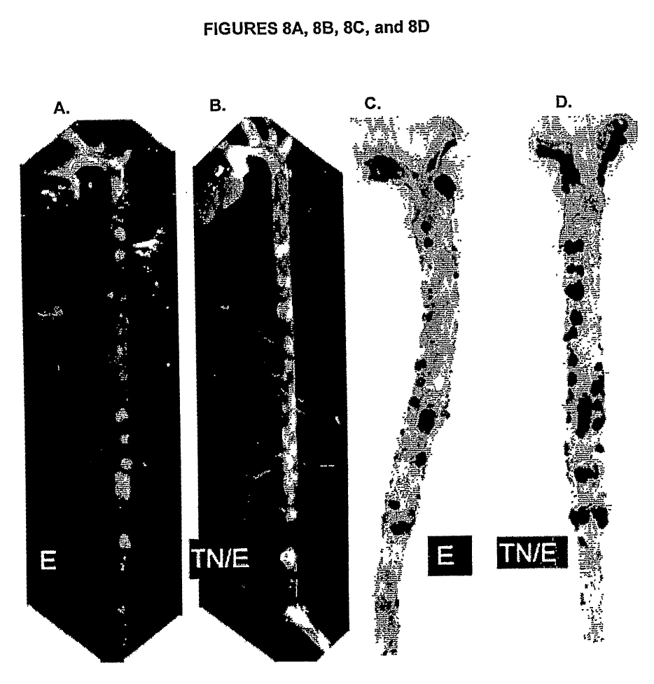

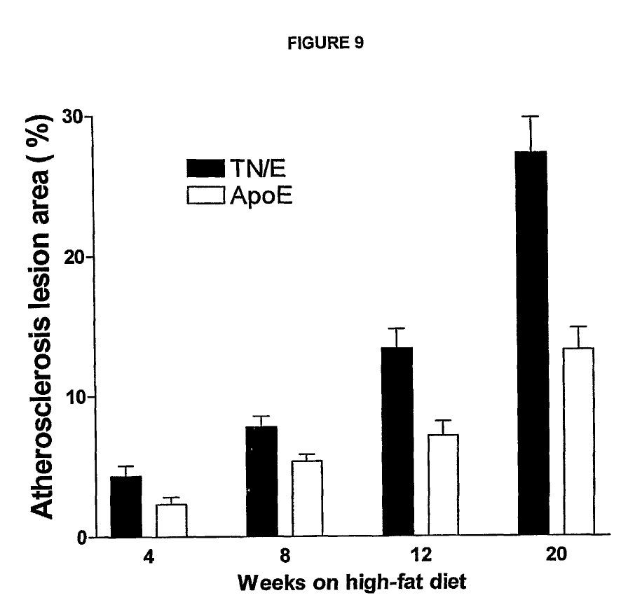

Next, the time-course of lesion development was examined in ApoE null and ApoE/TN null mice. Most (8/10) ApoE mice on high-fat diet for 4 weeks showed no visible plaques (FIG. 7A). In contrast, all TN/E mice fed high-fat diet for 4 weeks showed visible atherosclerotic lesions in the various regions of the aortic arch, especially in the innominate artery (FIG. 7B). Similarly, the atherosclerotic lesions in aortic arch of ApoE/TN null mice on high-fat diet for 8, 12, and 18 weeks were larger than those in ApoE null mice (not shown). In addition to the aortic arch, there were differences in the number and size of the lesions in the aortas of the two mouse genotypes (FIG. 8). As shown in FIG. 9, the aortic lesion area in ApoE null mice on high-fat diet for 18 weeks was 19±1.5% (n=10) vs. 32±1.9% in ApoE/TN null mice (n=10, p<0.05).

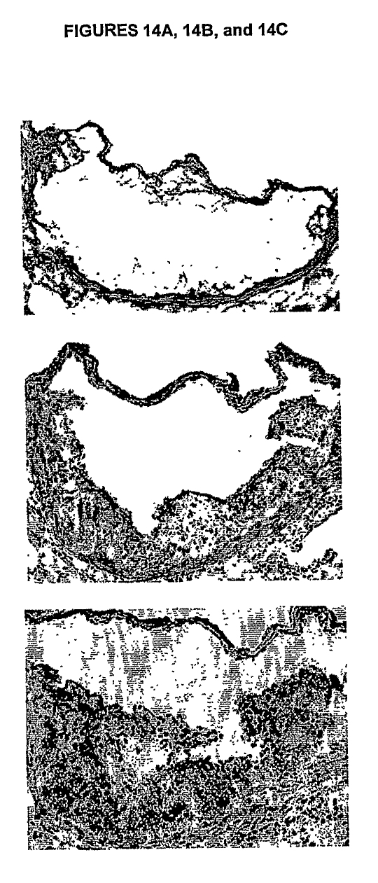

While little, if any, atherosclerotic lesions were detected in ApoE null mice on high-fat diet for 4 weeks (FIG. 14A), aortic sinus atherosclerotic lesions were found in all of the 10 ApoE/TN mice examined (FIG. 14B). No CD8, CD4, and CD5 immunoreactivity cells were detected in the lesions of ApoE and ApoE/TN null mice; however, small numbers of CD90.2-positive T cells were found in the adventitia of ApoE/TN null mice lesions (not shown). Moma-2 staining revealed an accumulation of a large number of macrophages in the aortic sinus lesions of ApoE/TN null mice on high-fat diet for 4 weeks (FIG. 14C). At 8 weeks on high-fat diet, the ApoE/TN null mice developed advanced lesions containing cholesterol clefts whereas lesions in ApoE mice were relatively small and did not have the characteristics of an advanced (not shown).

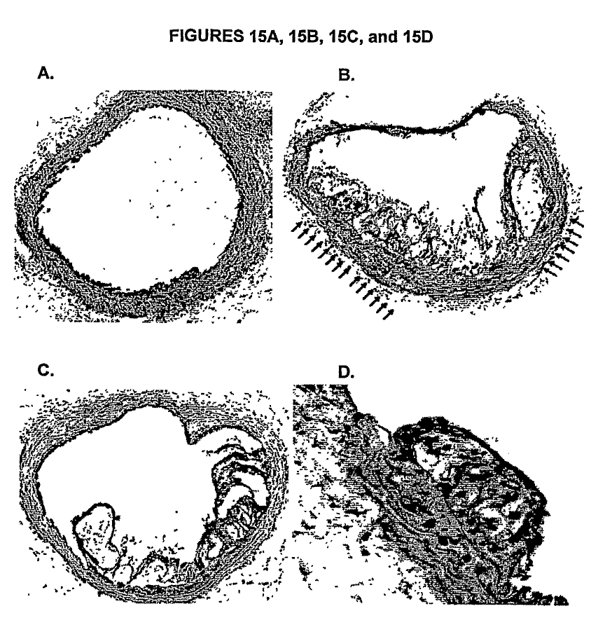

No lesions were detected (Oil red O positive) in the innominate artery of ApoE null mice on high-fat diet for 4 weeks (FIG. 15A). In contrast, atherosclerotic lesions were detectable at 4 weeks of high fat diet in ApoE/TN null mice (FIG. 15B). In fact, the innominate artery atherosclerotic lesions in ApoE/TN null mice on high-fat diet for 4 weeks were larger than those of ApoE null mice on high-fat diet for 18 weeks (FIG. 15C).

In addition to the lesion size and its complexity, atherosclerotic lesions in the aortic sinus and innominate artery showed focal medial erosion with plaque protrusion into the adventitia in the case of all ApoE/TN null mice on high-fat diet for 4 weeks (FIG. 15B, arrows). Higher magnification of these lesions shows that plaque cells infiltrated into the media of the aortic wall and degraded elastic lamina in an internal-to-external gradient (not shown). In contrast, the medial layer of atherosclerotic lesions of ApoE null mice remained intact and well defined even after 18 weeks on high-fat diet (FIG. 15C). The plaque size was not related to medial erosion, since small lesions found in the subclavian artery of ApoE/TN null mice on high-fat diet for 4 weeks also showed protrusion of intimal cells and erosion of the media (FIG. 15D).

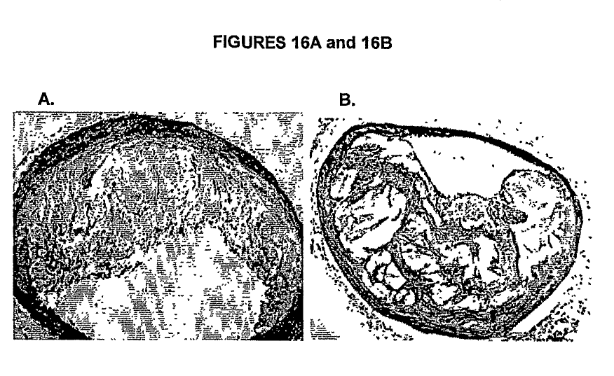

SMCs (SM-α-actin positivity) were detected in the media of normal arteries of both ApoE and ApoE/TN null mice (not shown). In the ApoE null mice on high-fat diet for 18 weeks, the media of the uninvolved region of innominate artery as well as media underlying the lesions exhibited α-actin positivity (FIG. 16A). In contrast, while the uninvolved media of innominate artery of TN/E mice stained positive for α-actin, medial cells underlying the plaque exhibited significantly reduced positivity in all of the ApoE/TN null mice examined (FIG. 16B). Similar results were obtained when the frozen sections from aortic sinuses were stained with the anti-α-actin antibody (not shown). While not wishing to be bound by any theory, these data suggest that the absence of TN is selectively associated with a marked reduction in SMCs positivity in the atherosclerotic lesion containing segments.

To determine whether the differences in lesion development between the two mouse genotypes are related to their lipoprotein profile, the total lipoprotein, serum cholesterol, and triglyceride levels was measured in plasma pooled from the ApoE and ApoE/TN null mice on high-fat diet (5 male and 5 female in each group). Ingestion of an atherogenic diet for 4 weeks led to severe hypercholesterolemia in both mouse genotypes. The average total plasma cholesterol levels for ApoE/TN and ApoE null mice (10 mice/group/time point) are shown in FIG. 127. On a high-fat diet, the cholesterol levels increased to 1240±420 mg/dl and 1290±510 mg/dl for ApoE/TN and ApoE null mice respectively. There was no significant difference in the serum cholesterol levels between ApoE/TN and ApoE null mice on high-fat diet for 4-12 weeks. Serum cholesterol levels of the ApoE/TN null mice on high-fat diet for 18 weeks were reduced by 30% when compared to ApoE null mice. Wild type C57BL/6 mice (“C57”) were used as negative control.

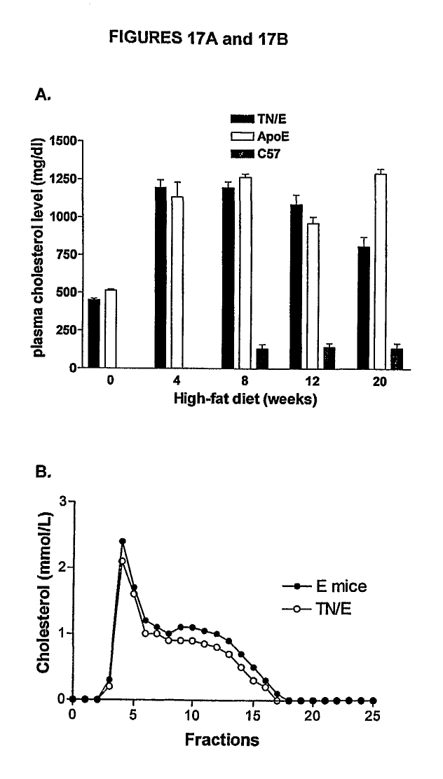

Size exclusion chromatography was performed on serum samples from individual mice to determine whether TN deficiency altered serum lipoprotein distributions. The majority of serum cholesterol was in the VLDL fraction, with no difference between the two mouse genotypes on high-fat diet for 4 weeks (FIG. 17B). Similar results were obtained using pooled plasma from ApoE/TN and ApoE null mice on high-fat diet for 18 weeks (not shown). Similarly, no significant difference was found in the triglyceride levels between the two mouse genotypes. Triglyceride levels for ApoE/TN null mice on a chow diet were 175±150 mg/dl and ApoE null mice were 165±176 mg/dl. Triglyceride levels increased to 269±154 mg/dl for ApoE/TN null mice and 265±162 mg/dl for ApoE null mice on high-fat diet for 4 weeks, and these levels were not changed for the two mouse genotypes on high-fat diet for 18 weeks. While not wishing to be bound by any theory, these data suggest that TN deficiency does not alter lipoprotein metabolism in ApoE null mice.

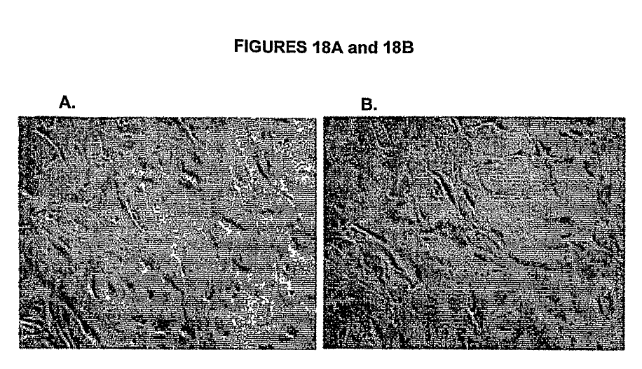

Past studies have implicated TN in the migration/proliferation/viability of cultured human and rat SMCs. Therefore, this research assessed whether TN deficiency affects the migration, viability, and growth rate of SMCs from the two mouse genotypes. SMCs migrated out of the aortic explant of ApoE/TN null mice, remained viable, and replicated in culture (FIG. 18B) similar to cells isolated from ApoE null mice (FIG. 18A). No differences were found in the growth rate and saturation density between the SMCs isolates from ApoE null and ApoE/TN null mice (not shown). While not wishing to be bound by any theory, collectively, this data suggests that SMCs may not be the target of TN activity in vitro and in vivo.

### Expression of TN in Lesions in ApoE Null Mice on a High Fat Diet.

ApoE null mice were placed on high-fat diet (10 mice/group) for 4 (FIG. 6A), 8 weeks, (FIG. 6B), and 20 weeks (FIG. 6C). The mice were sacrificed, their aortic sinuses were collected, snap-frozen, embedded in OCT, and frozen sections (10 μm) were used for immunostaining using 1:200 dilution of anti-mouse TN antibodies provided by Dr. Erickson (Duke University). The sections stained with unrelated, isotype-matched secondary antibody served as negative control (not shown). Original magnification 20×.

### Distribution of Lesions in the Aortic Arch of ApoE/TN Null Mice.

Representative photographs of the gross appearance of the aortic arch from ApoE (FIG. 7A) and ApoE/TN null mice (FIG. 7B). The animals were placed on high-fat diet for 4 weeks and then sacrificed. The thoracic cavity was opened; the aortic arch as well as the aorta were exposed, and photographed under a dissecting microscope with appropriate back lighting. (FIG. 8) The two left panels show the distribution of aortic lesions from each mice genotype on high-fat diet for 18 weeks. The two right panels show the Oil red O staining of the aortas. FIG. 9 shows the relative lesion area in ApoE/TN and ApoE mice on high-fat diet for 4-20 weeks.

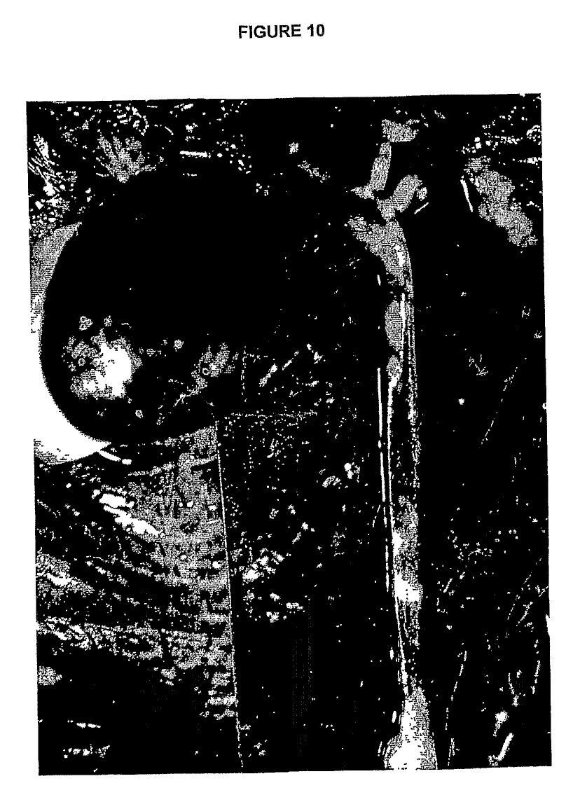

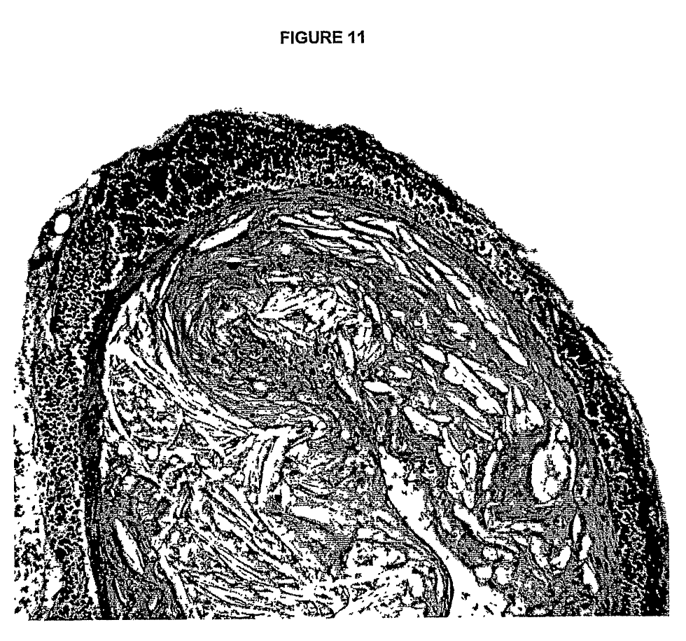

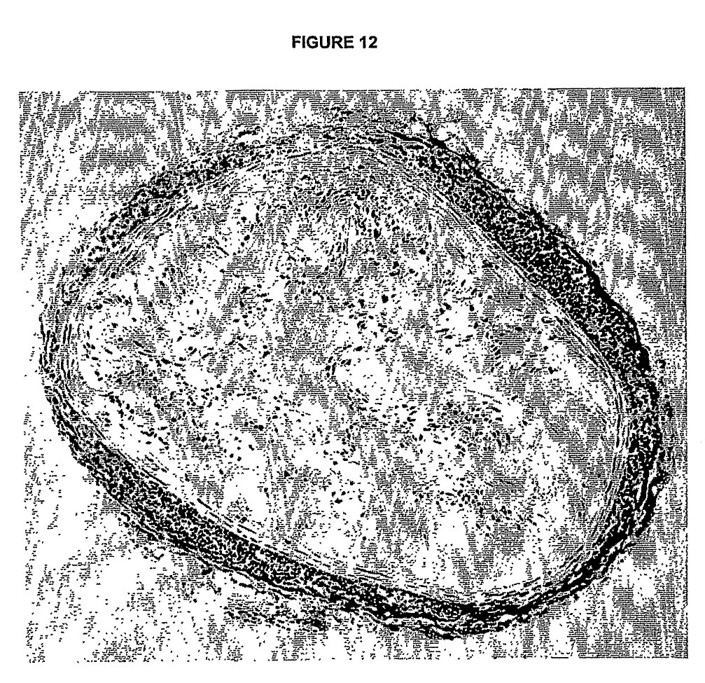

FIG. 10 shows the aortic arch from ApoE/TN null mice placed on high-fat diet for 30 weeks and then sacrificed. The aortas were harvested, opened, stained with Oil red O and lesion area was measured. Comparison of the percentage of the atherosclerotic lesion to total aorta area between two genotypes (p<0.05). Values are the mean ±SD of 10 mice/group/time point. FIG. 11 shows the H&E staining of aortic lesion of TN/E mice fed a high-fat diet for 30 weeks at 20× magnification. FIG. 12 shows the toluidine blue staining of aortic lesion of TN/E mice fed a high-fat diet for 30 weeks at 10× magnification.

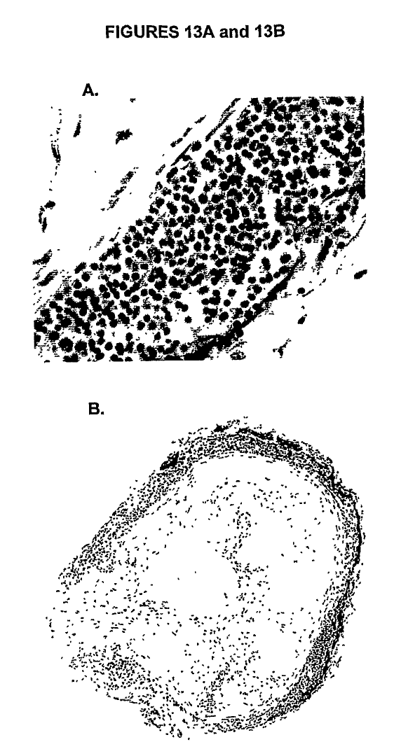

FIG. 13A shows mast cell accumulation in the aorta of TN/E mice fed a high-fat diet for 30 weeks at 100× magnification. FIG. 13B shows mast cell accumulation in the aorta of TN/E mice fed a high-fat diet for 30 weeks at 10× magnification.

### Lesion Development in the Aortic Sinuses of Mice on a High-Fat Diet for 4 Weeks.

Frozen sections from the aortic sinuses of ApoE null (FIG. 14A) or ApoE/TN null (FIG. 14B) mice were stained with Oil red 0 or Moma-2. FIG. 14C shows macrophage distribution in the ApoE/TN null mice on high-fat diet for 4 weeks. The images are obtained at 20× magnifications.

### Lesion Development in the Innominate Artery of ApoE and ApoE/TN Null Mice.

FIGS. 15A and 15B show representative Oil red O staining of the frozen sections from innominate artery of ApoE and ApoE/TN null mice on high-fat diet for 4 weeks, respectively. FIG. 15A shows Oil red O staining of frozen section from artery of ApoE null mice on high-fat diet for 18 weeks. FIG. 15D shows a representative Oil red O staining of left subclavian artery from ApoE/TN null mice on high-fat diet for 4 weeks. Compare erosion of media in lesions from ApoE/TN null mice on high-fat diet for 4 weeks (FIG. 15B, arrow) with those of ApoE null mice on high-fat diet for 18 weeks (FIG. 15C).

### Distribution of SMCs in the Innominate Artery Lesions of ApoE and ApoE/TN Null Mice.

Frozen sections from the innominate artery of ApoE null mice (FIG. 16A) and ApoE/TN null mice (FIG. 16b) on high-fat diet for 18 weeks were stained with 1:50 dilution of monoclonal antibody to smooth muscle α-actin.

### Cholesterol-Lipoprotein Distribution in ApoE and ApoE/TN Null Mice.

FIG. 17A shows total serum cholesterol measured in serum from 5 individual ApoE and ApoE/TN null mice on high-fat diet for 4-20 weeks. FIG. 17B shows lipoprotein-cholesterol distribution measured in serum from 5 individual ApoE and ApoE/TN null mice using superose-6 chromatography.

Culture of Aortic SMCs from ApoE/TN Null Mice.

Aortic SMCs from ApoE (FIG. 18A) and ApoE/TN (FIG. 18B) null mice were cultured as described. Cultured cells from ApoE/TN null mice remained viable and proliferated in the complete absence of TN.

### Example 3

### Genetic Ablation of TN Destabilizes Atheroma in Apo E Mice and Leads to Elevated Serum Eotaxin Levels

It was noted that chronic hyperlipidemia leads to development of unstable lesions in TN/E group, but not ApoE mice. To delineate the underlying mechanism responsible for the development of unstable plaques in TN/E mice, the entire known mouse inflammatory protein profile was examined.

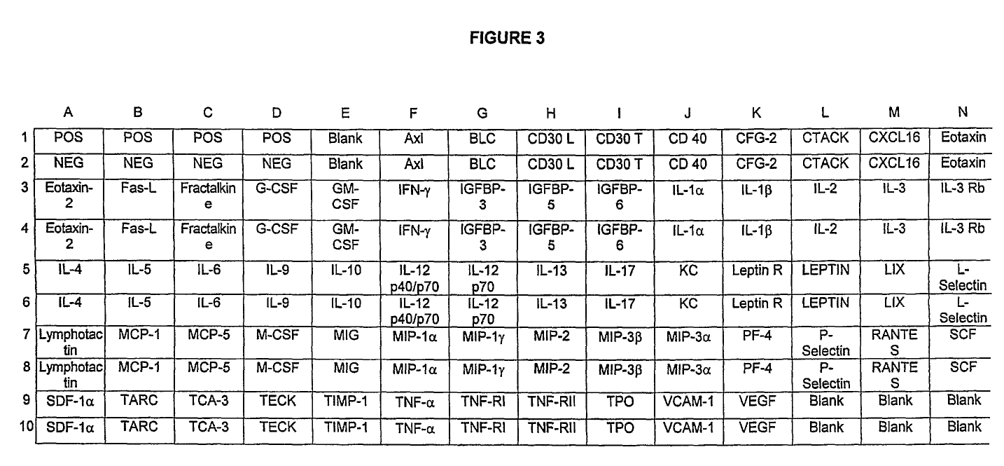

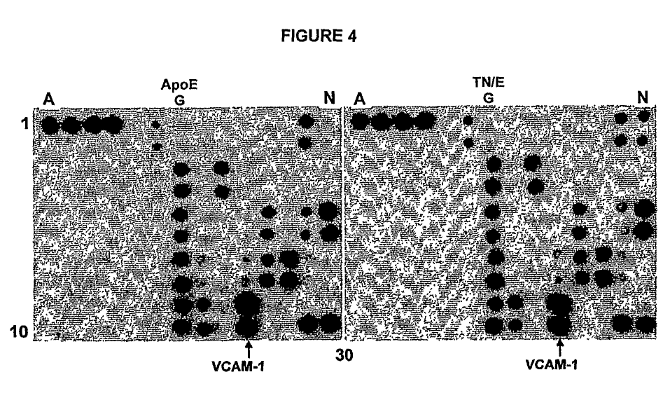

It was theorized that the differences in atherosclerosis between ApoE mice and TN/E group are due to distinct inflammatory responses in the two genotypes. To test this, plasma from TN/E and ApoE mice on a high-fat diet for 24 weeks was tested with an antibody array representing 62 known mouse inflammatory proteins. The array was purchased from RayBio (Norcross, Ga.). It is based on an antibody sandwich assay where antibodies to the inflammatory proteins are spotted in duplicate onto a membrane. Each membrane contains 6 positive control spots, 4 on the upper left (1A-D) and 2 on the lower right (10M10N). The plasma from each group of mice is diluted and incubated with a membrane. This is followed by incubating each membrane with a cocktail of biotin-labeled antibodies. The bound antibodies were visualized with HRP-conjugated streptavidin. All reagents required for this experiment are included in this kit. The experiment was repeated three times with three different membranes using plasma from different pools of TN/E group and ApoE group. All experiments yielded identical results. The template and representative membrane images from ApoE group and TN/E group on a high-fat diet for 24 weeks are shown in FIG. 3 and FIG. 4 respectively.

To produce these data, the membranes were processed essentially as described by the manufacturer. Briefly, membranes were blocked by addition of a blocking buffer and incubated at room temperature for 1 hr. Membranes were then incubated with 1 mL diluted plasma (diluted 1:1 with PBS) from 6 ApoE groups or 6 ApoE/TN groups (6 mice/group, ˜100 L/mice) at 4° C. overnight. After washing, 1 mL of biotin-conjugated antibodies was added to each membrane and then incubated at room temperature for 1 hr. After washing, 2 mLs of diluted HRP-conjugated streptavidin were added to each membrane and then incubated at room temperature for 1 hr. After washing, the membranes were developed by addition of buffers C and D, at room temperature for 1 hr. The membranes were exposed to x-ray film and the identity of the Spots was determined using the template (FIG. 3).

No difference in the expression pattern of cytokines/chemokines wad found between the two groups of mice on a high-fat diet for 4 weeks (not shown). However, the expression pattern of mice groups on a high-fat diet for 24 weeks was different (FIG. 4). The following spots were detected in the arrays (from left to right, top to bottom): GNFRII (H9H10), IGFBP-6 (1314), SOLUBLE VCAM-1 (J9J10), Leptin R (K5K6), PF4 (K7K8), soluble p-selectin (L7L8), CXCL16 (M1M2), LIX (M5M6), and soluble L-selectin (N5N6), positive controls (M10N10). Modulation of the expression of known proinflammatory cytokines (IFN, TNF-a, IL-1 to IL-17, CSF-G, CSF-GM, and CSF-M, etc.), chemokines (MIPs, MCPs, MIG, fractalkine, CTACK, CD40, CD30, etc.), and factors involved in angiogenesis (VEGF and Leptins) was not detected. Eotaxin (N1N2) was the only molecule that was consistently over-expressed in the plasma of the TN/E group when compared to the ApoE group. Collectively, these data demonstrate that eotaxin is the only pro-inflammatory molecule that is selectively up-regulated in the plasma of chronic hyperlipidemic TN/E mice. In FIG. 4, VCAM-1 was labeled as a reference point.

### Example 4

### Measurement of Plasma Eotaxin Levels by ELISA

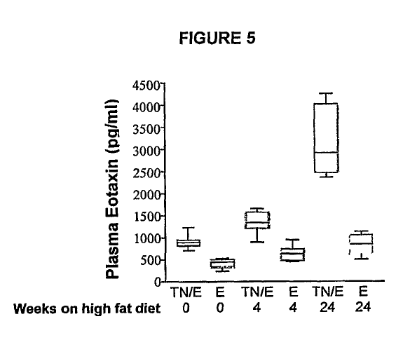

To confirm and expand the array findings in Example 2, plasma levels of eotaxin in the ApoE group and the TN/E group on high fat diet for 4 and 24 weeks were determined by an ELISA assay (FIG. 5). Plasma from 6-weeks old mice groups prior to feeding a high-fat diet was used as controls (zero week groups). Eotaxin levels of TN/E and ApoE groups before initiation of high-fat diet feeding was 908.3±40.05 (n=12) and 421.7±27.55 (n=15, P<0.0001), respectively (FIG. 20). At 4 weeks on a high-fat diet, the level of eotaxin for the TN/E group was 1357±62.41 (n=14) and for the ApoE group was 649.7±50.35 (n=10, P<0.0001). At 24 weeks on high-fat diet, the eotaxin level for TN/E group was 3170±216.0 (n=12) and for the ApoE group was 839.0±92.09 (n=11, P<0.0001). The differences in the eotaxin level between the ApoE groups before and after high-fat diet (4 and 24 weeks) were not statistically significant (P>0.05), as determined by Bonferroni's Multiple Comparison Test. In contrast, differences in eotaxin levels between the TN/E groups before and after high-fat diet were statistically significant (0 and 4 weeks, P<0.05; 4 weeks and 24 weeks, P<0.001). Overall, the level of eotaxin in TN/E groups was significantly higher than those in ApoE groups, even in the levels in TN/E groups were markedly increased with chronic hyperlipidemia. Further, the eotaxin levels in the TN/E groups were markedly increased with chronic hyperlipidemia.

### Example 5

### Statistical Analysis

All data are represented as mean ±SEM. Statistical analysis was performed by the Student t test. If data were nonparametric, they were analyzed by the Mann-Whitney rank sum test. All data analyses were performed with the use of SigmaStat 2.03 software (SPSS, Inc). Values with P<0.05 were considered statistically significant.

While the description above refers to particular embodiments of the present invention, it will be understood that many modifications may be made without departing from the spirit thereof. For example, alternate methodologies and procedures well known to those of skill in the art may be substituted for the ELISA procedure described in connection with the invention. Such alternate methodologies and procedures may be readily implemented without undue experimentation. The presently disclosed embodiments are therefore to be considered in all respects as illustrative and not restrictive.

What is claimed is:

1. A method for detecting the presence of atherosclerosis in a mammalian subject, comprising:
(a) obtaining a sample of serum from a mammalian subject;
(b) contacting the serum with an eotaxin binding partner, whereby an eotaxin/eotaxin binding partner complex is formed;
(c) detecting the eotaxin/eotaxin binding partner complex, wherein the level of eotaxin/binding partner complex correlates with the eotaxin serum level of the subject; and
(d) correlating the serum level of eotaxin with the presence of atherosclerosis.

2. The method of claim 1, wherein the binding partner is selected from the group consisting of peptides, antibodies, small molecules, and combinations thereof.

3. The method of claim 1, wherein the binding partner is detectably labeled.

4. The method of claim 1, wherein the binding partner is an antibody.

5. The method of claim 1, wherein the detecting of the eotaxin/eotaxin binding partner complex is accomplished by enzyme-linked immunosorbent assay (ELISA).

6. The method of claim 1, wherein the detecting of the eotaxin/eotaxin binding partner complex is accomplished by antibody array.

7. The method of claim 1, wherein the eotaxin/eotaxin binding partner complex is detected by a second binding partner.

8. A method for diagnosing atherosclerosis in a mammal, comprising:
(a) obtaining a sample of serum from a mammalian subject;
(b) contacting the serum sample with an eotaxin binding partner capable of forming a complex with eotaxin;
(c) detecting the presence of the eotaxin/eotaxin binding partner complex, wherein the concentration of the eotaxin/eotaxin binding partner complex correlates with the concentration of eotaxin in the serum of the mammalian subject, and wherein a concentration of eotaxin greater than about 160 pg/mL indicates that the mammal has atherosclerosis.

9. A kit for the diagnosis of atherosclerosis in a mammalian subject, comprising:
(a) a solid substrate having immobilized thereon a first anti-eotaxin antibody;
(b) a second anti-eotaxin antibody reactive with the first anti-eotaxin antibody, wherein the second anti-eotaxin antibody is detectably labeled;
(c) instructions for performing an enzyme-linked immunosorbent assay (ELISA) for the presence of eotaxin in a sample of serum obtained from the mammalian subject; and
(d) one or more reagents for performing the ELISA.

10. The kit according to claim 9, further comprising a multi-well microtiter plate, and wherein the first antibody is immobilized in the wells of the multi-well microtiter plate.

11. The kit according to claim 9, wherein the second antibody is fluorescently-labeled.
